(19) United States
(12) Plant Patent Application Publication
(10) Pub. No.: US 2004/0083508 P1
(43) Pub. Date: Apr. 29, 2004

(54) European plum named '92-110-69'

(75) Inventor: Timothy F. Bourne, Visalia, CA (US)

Correspondence Address: JONDLE & ASSOCIATES P.C., 9085 EAST MINERAL CIRCLE, SUITE 200, CENTENNIAL, CO, 80112, US

(73) Assignee: Sunview Vineyards

(21) Appl. No.: 10/280,571

(22) Filed: Oct. 24, 2002

(51) Int. Cl. A01H 5/00

(52) U.S. Cl. PLT/184

(57) ABSTRACT

A new and distinct variety of European plum (Prunus domestica) is described herein. The new variety ripens in mid-season with large, red-purple fruit and firm flesh, with a tendency to produce some double fruits. Ripened fruit attains a very high level of sugar and holds in cold storage very well.

### EUROPEAN PLUM NAMED ‘92-110-69’

### Genus and Species

Prunus domestica

### BACKGROUND AND SUMMARY OF THE INVENTION

The invention described here is a new variety of plum, Prunus domestica cv. ‘92-110-69’. The seedling originates from a planned cross in 1993 in Tulare County, California. The new variety is characterized by have large, red-purple, elliptic fruits which ripen in early August in the Delano, Calif. area. The high quality fruit has firm texture, good storage characteristics, and is very sweet, making it suitable for the fresh market.

The emasculated seed parent was the variety ‘Empress’ (unpatented) and the pollen parent was the variety ‘Moyer Perfecto’ (unpatented). Seed from the planned cross was harvested in the summer of 1993, stratified, then planted in the spring of 1994 in Kern County near the city of McFarland. The original seedling tree was selected in 1997 and was then propagated to a two tree plot. In 1998, a forty tree plot was propagated from wood of the two tree plot and the original seedling tree. Observations on the original seedling tree and subsequent propagations indicated that the fruit of the tree was suitable for shipment where the fruit has received commercial acceptance.

### COMPARISON WITH PARENTAL CULTIVAR

The new variety is intermediate in its characteristics between its two parents. It ripens about one week before ‘Moyer Perfecto’ in the Delano area. It has the higher productivity characteristics of the ‘Empress’ variety as well as better adaptation to the hot climate of the southern Central Valley of California.

### DESCRIPTION OF THE FIGURES

The accompanying drawings illustrate the following:

FIG. 1. Harvested fruit.

FIG. 2. Fruit hanging on the tree.

FIG. 3. Leaves of the variety.

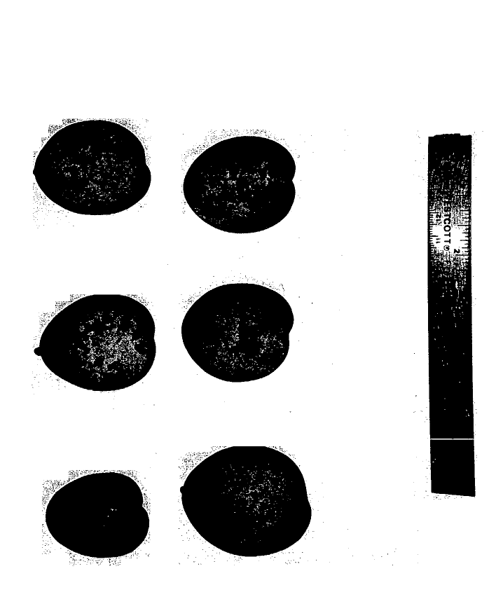

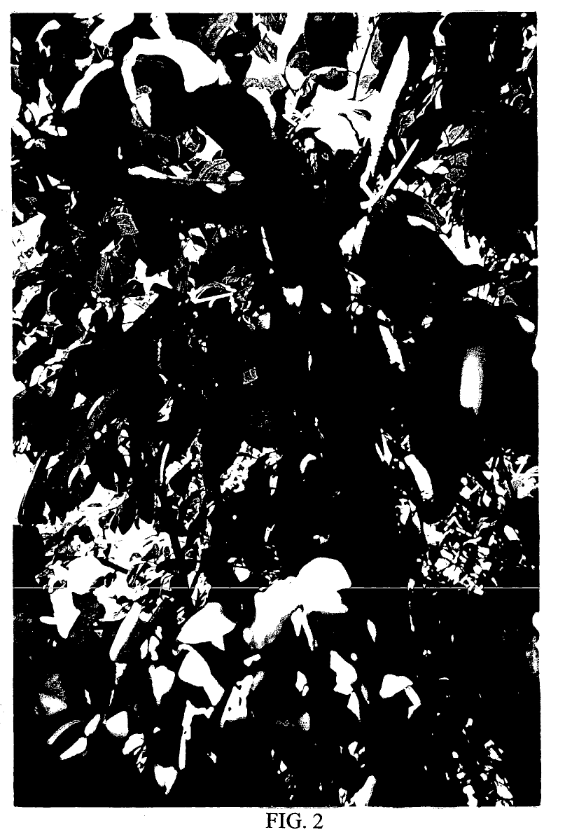

### DETAILED BOTANICAL DESCRIPTION OF THE INVENTION

The following description of European Plum ‘92-110-69’ contains references to color names taken from the Munsell Color Chart for Plant Tissues, published by Musnsell Color, New Windsor, N.Y.

Tree:

Habit.—Upright.

Size.—Large, very vigorous.

Productivity.—Very good, sets excessively heavy crop requiring thinning.

Rootstock.—Myrobalan 29C.

Trunk:

Diameter.—Typical of plums grafted onto Myrobalan 29C.

Bark texture.—Very rough.

Trunk color.—Grayish brown: 5YR 5/2 to 6/2.

Trunk lenticel.—color Orangish: 5YR 5/8.

Trunk lenticel length.—4 mm.

Trunk lenticel number.—Variable. Large sectors with no lenticels to as many as 6 per sq.cm.

Branches:

Size.—Typical for European plum trees depending on pruning practices.

Color 5YR 3/2 to ¾.

Lenticels.—About 1 mm in diameter; approximately 36 per square cm.

Lateral branching on first year branches.—uncommon.

Leaves:

Blade length.—8.5 cm.

Blade width.—5.5 cm.

Petiole length.—1.8 cm.

Leaf shape.—Obovate to elliptic.

Leaf margins.—Crenate, margins of upper surface cupped downward.

Teeth.—Both sides generally convex; about 2 mm in length.

Upper surface color.—Dark green, 7.5GY 4/4 to 4/6.

Lower surface color.—Dark green, 7.5 GY 5/6 to 5/8.

Erect hairs on veins of lower surface.—numerous.

Erect hairs on upper surface.—absent.

Erect hairs on upper surface of petiole.—Sparse.

Flowers:

Date of flowering.—Mar. 24, 2001.

Color.—White.

Petal length.—10 mm.

Sepal length.—7 mm.

Anther color.—Yellow, 5Y 8/12.

Fruit:

Harvest maturity.—Mid-season, Aug. 10, 2001

Skin color.—Reddish purple, 5RP 10/6 to 10/8.

Flesh color.—Yellow: 2.5Y 7/10.

Length.—4.6 cm.

Width.—4.1 cm.

Fruit size.—Large, 52.2 g.

Refractometer test.—23.2 brix.

Pressure at harvest (penetrometer test).—13.8 lbs.

Configuration.—Elliptic.

Apex.—Rounded.

Pistil point.—Smooth.

Skin thickness.—Thin, typical of European plum.

Texture.—Smooth.

Tendency to crack.—None.

Fruit abnormalities.—Some tendency to produce double fruit.

Juiciness of flesh.—Very juicy.

Flavor.—Mild and sweet, typical of European plum.

Aroma.—Mild.

Texture.—Crisp.

Fibers.—None.

Ripening.—Uniform throughout the fruit.

Eating quality.—Good.

Use.—Fresh market.

Keeping quality.—Good.

Shipping quality.—Good.

Stone:

Stone adherence to flesh.—Clingstone.

Stone shape.—Elliptic coming to a very point at apex.

Fibers.—None.

Surface.—Rough.

Color.—Brown: 5YR 5/8 to 5/10.

Length.—2.8 cm.

Width.—1.5 cm.

Thickness.—0.8 cm.

Disease resistance: Typical of European plums; no problems seen in growing trials.

What is claimed is:

1. The new and distinct variety of EUROPEAN PLUM cv. ‘92-110-69’ herein described and illustrated and identified by the characteristics enumerated above.
